(19) United States
(12) Patent Application Publication
(10) Pub. No.: US 2020/0121766 A1
(43) Pub. Date: Apr. 23, 2020

(54) PHARMACEUTICAL COMPOSITION COMPRISING PANCREATIN AND A LIPASE-CONTAINING COATING

(71) Applicant: Nordmark Arzneimittel GmbH & Co. KG, Uetersen (DE)

(72) Inventors: Richard Fuhrherr, Uetersen (DE); Jan Lüdemann, Uetersen (DE); Jörn Tonne, Uetersen (DE)

(21) Appl. No.: 16/490,547

(22) PCT Filed: Mar. 5, 2018
(86) PCT No.: PCT/EP2018/055351

§ 371 Date: Sep. 1, 2019

(30) Foreign Application Priority Data
Mar. 3, 2017 (DE) 10 2017 104 482.5

(51) Int. Cl. A61K 38/46; A61K 9/28

(52) U.S. Cl. CPC A61K 38/465; A61K 9/2866; A61K 9/2873

(57) ABSTRACT

The present invention relates to a pharmaceutical composition comprising pancreatin, which has a coating that contains a least one lipase. Preferably the at least one lipase is burlulipase. It also relates to a medicinal product comprising such a pharmaceutical composition. A method of producing such a pharmaceutical composition also forms part of the present invention.

### INTRODUCTION

The present invention relates to a pharmaceutical composition comprising pancreatin, which has a coating that contains a least one lipase. Preferably, the at least one lipase is burlulipase. It also relates to medicinal products comprising such a pharmaceutical composition. A method of producing such a pharmaceutical composition also forms part of the present invention.

### PRIOR ART

The pancreas is a gland with both an endocrine and exocrine effect. It produces digestive enzymes that are released into the duodenum (exocrine gland) and in the intestines break down fats, carbohydrates and proteins in food into a form than can be absorbed by the intestinal mucous membrane. Usually designated as “digestive enzymes” are enzymes which preferably originate from the three enzyme classes that are required for the digestion of the three basic components of food—lipases for fats, amylases for carbohydrates, proteases for proteins. As such, in healthy persons these are contained in sufficient quantities in the exocrine pancreatic secretion.

A disorder which affects the exocrine portion is pancreatitis, for example. In pancreatitis, or inflammation of the pancreas, the released digestive enzymes lead to self-digestion of the organ and hence to a severe inflammatory reaction. The inadequate production of pancreatic enzymes is known as exocrine pancreatic insufficiency. It is a decrease in the production of digestive enzymes, as a result of which food can no longer be broken down to a sufficient degree. This can, for example, be acquired through the loss of pancreatic tissue in chronic pancreatitis or pancreatic cancer, but can also be congenital in the case of genetically-caused disorders such as cystic fibrosis. Exocrine pancreatic insufficiency leads to digestive problems with steatorrhoea (fatty stools) and other symptoms and is generally treated through the administration of pancreatin with meals.

Cystic fibrosis is an inherited autosomal recessive metabolic disorder in which the composition of all exocrine gland secretions is altered. Due to a modified gene on chromosome number 7 (CFTR gene), cellular salt and water transportation is disrupted. Therefore, for example, the digestive juices produced by the pancreas are more viscous than usual and block the gland's excretory ducts. The build-up of digestive juices leads to irritation and eventually to damage to the pancreas. Furthermore, through the lacking digestive enzymes in the intestine the absorption of nutrients is made more difficult. The consequences are malnutrition and growth disorders. Exocrine pancreatic insufficiency induced by cystic fibrosis is usually treated with known enzyme therapy through the administration of pancreatin.

The active substance “pancreatin”—in the European Pharmacopoeia (Ph. Eur.) monographed as “pancreas powder”, in the US Pharmacopoeia (USP) as “pancreatin” or “pancrelipase”—is obtained through extraction from porcine pancreas and contains a mixture of active digestive enzymes. The main components of pancreatin are lipases, amylases and various proteases. The most important therapeutic components of pancreatin are the lipases which break down dietary fats, improve the patient's nutritional condition and at the same time help to prevent unpleasant side effects of poor fat digestion, such as fatty stools.

However, the specific enzyme activities, in particular those of the pancreatic lipases, are relatively low. For the patients, in therapeutic practice this results in the unpleasant requirement of having to swallow a considerable number of generally very large pancreatin-containing drug formulations with every meal. This is a fundamental burden in itself, which together with the already serious disorders leads to further restrictions in the quality of life of the patients. In the case of patients who cannot or do not want to swallow the large drug formulations, including patients being artificially fed, small children, infants and older patients, there are considerable additional obstacles to administration. Crushing the solid formulations, which is otherwise the established option in such cases, must be eschewed here, as it leads to the destruction of the protective gastric juice-resistant film, so that the enzymes are delivered unprotected to the acidic gastric juices and can be inactivated in this environment.

In these cases the capsules, which contain for example pancreatic enzyme preparations, are opened and the solid, gastric juice-resistantly coated multiparticulate units present therein are scattered onto or into the meal, e.g. apple purée. During such oral intake the integrity of the functionally gastric juice-resistant coating can be destroyed by chewing and the enzymes can be released, denatured and thus rendered inactive at the wrong point, especially before passing through the stomach. In addition, individual multiparticulate units can remain stuck in the cheek pockets and there result in irritation and damage to the mucous membranes, in the worst case causing ulceration.

Furthermore, pancreatic lipases are known to be acid-labile. Therefore, pancreatin-containing medicinal products are generally provided with a gastric juice-resistant coating to protect the enzymes from gastric acid. The generally available forms of administration of pancreatin are usually gastric juice-resistant film-coated tablets, micro-tablets, micro-pellets/granulates, micro-dragées and capsules, as well as powder. After passing through the stomach and with the increase in the pH value on entering the small intestine, the gastric juice-resistant protective film dissolves and releases the active substance which can then take effect in the food bolus. Medicinal products containing pancreatin must be taken with meals so that they reach the small intestine together with the consumed food in order to be able to work there.

To improve the efficacy of this known therapy, it is usual and frequently necessary to give the patients additional acid-suppressing medications, such as, for example, proton pump inhibitors (PPIs) or H2-receptor antagonists in order to inhibit gastric acid secretion. In the stomach, and subsequently in the intestinal lumen, these bring about a higher pH value and thus lead to better active substance release from the in general functionally gastric juice-resistantly coated pancreatin products. However, in long-term use acid-suppressing medications have considerable side effects, sometimes with chronic damage, such as the occurrence of osteoporosis or an increased risk of myocardial infarction.

The use of lipases other than pancreas lipase has already been proposed. WO 2010/085975 A1 discloses liquid formulations of burlulipase for the treatment of digestive disorders, in particular pancreatitis and cystic fibrosis. Burlulipase (international non-proprietary name; INN) is the lipase of bacterial species Burkholderia plantarii. Burlulipase is a triacylglycerol acylhydrolase (EC 3.1.1.3), which has an amino acid sequence corresponding with the lipases produced by Burkholderia plantarii and Burkholderia glumae. Burlulipase is produced by way of a classic fermentation process in which Burkholderia plantarii, a non-recombinant, gram-negative bacterium is used as the production strain. Pure burlulipase can exhibit a specific activity of more than 3,500 TBU/mg (tributyrine units per milligram of protein). Due to this high lipolytic activity, burlulipase is particularly suitable for supporting digestive performance in healthy and sick people. Burlulipase can be contained in very high active substance densities with high specific activity, so that only small quantities of substance (i.e. a small mass or small solution volume) have to be administered. However, burlulipase has drawbacks which hitherto have prevented its practical use in medicinal products. It is unstable under various conditions. For example, the activity decreases in uncooled liquids during storage and the tabletting of compositions containing burlulipase leads to an unacceptable loss of activity (unpublished results).

### OBJECT OF THE PRESENT INVENTION

The task of the present invention is to provide medicinal products in solid form which are suitable for the prevention and/or treatment of digestive disorders, in particular for the prevention and/or treatment of exocrine pancreatic insufficiency. This includes the treatment of patients with cystic fibrosis. In particular, these medicinal products should make improved prevention and/or therapy of these disorders possible. In particular, the additional administration of medications to inhibit gastric acid secretion or to regulate the acid in the stomach and/or in the duodenum should become unnecessary, or at least their administration should be able to be reduced. In addition, the medication should be suitable for administration to patients who have difficulty swallowing large drug formulations, such as small children, infants and older people. It should also be possible to mix the formulation for administration with food without the efficacy being impaired by this.

### DESCRIPTION OF THE INVENTION

### Pharmaceutical Compositions

One aspect of the present invention is a pharmaceutical composition comprising pancreatin, characterised in that it has a coating that contains at least one lipase. Preferred here is a pharmaceutical composition comprising pancreatin that has a coating containing at least one lipase, wherein the pharmaceutical composition comprises a core containing pancreatin onto which a coating is applied, characterised in that the at least one lipase used in the coating is a lipase different from the lipases present in the pancreatin. The effect that such a composition has compared with the conventional administration of pancreatin is that the quantity of lipase in the coating can be selected in such a way that the combined activity of the lipases in the pharmaceutical composition is sufficient to cover the lipolytic activity requirement. The lipolytic activity of pancreatin is frequently inadequate and has to be assured through the administration of large quantities of pancreatin or the additional administration of medications. Nevertheless, not infrequently even the administration of the largest possible quantity of pancreatin does not ensure sufficient lipolytic activity. This problem can be solved through the use of at least one further lipase which is applied in the form of a coating.

Lipases, proteases and amylases are present in pancreatin in predefined ratios. The pancreatin is generally and as far as possible dosed so that the patient is provided with sufficient lipases. As proteases and amylases are generally present in excess in pancreatin, these are often overdosed. In the present pharmaceutical composition, with the administration of smaller quantities (masses) of pancreatin in combination with at least one additional lipase, the ratio of lipases to proteases and amylases can be optimally adjusted and the adequate provision of the patient with lipases, proteases and amylases can be guaranteed. The absolute quantity (mass) of the medicinal product is thereby reduced. This increases compliance and the success of the treatment and also allows successful administration in the case of small children, infants and older and bedridden patients as well as patients who have difficulty taking large quantities of medicinal products. Additionally, the spatial separation of lipase from pancreatin means that the activity of the lipase is not influenced by the enzymes of the pancreatin. In particular, breakdown through proteases does not take place. On the other hand, due to the use of a coating in contrast, for example, to a mixture of particles, separation of the pancreatin and the at least one lipase in the coating does not take place.

The activity of the at least one lipase in the coating is preferably stable against the effect of gastric acid in vivo. In particular, it is preferably more stable against the effect of gastric acid than the lipases of the pancreatin. The at least one lipase used in the coating is preferably a lipase different from the lipases present in the pancreatin. Particularly preferably it is a microbial lipase. Microbial lipases can be easily produced on a large scale and it can be guaranteed that they do not contain any microbial contaminants harmful to humans. Very particularly preferably the at least one lipase is a bacterial lipase. As a rule, bacterial lipases have a greater lipolytic activity than pancreatin and are more acid-stable.

Very particularly preferably the at least one lipase is burlulipase. Because of the high specific activity of burlulipase, the use of a small quantity (mass) of burlulipase relative to the quantity of pancreatin is sufficient to assure adequate lipolytic activity. Furthermore, the activity of burlulipase is resistant to the effect of gastric acid. The production of solid formulations of burlulipase, such as tablets, is generally associated with a large loss of activity of the burlulipase. Typically only around 60% of the original activity (measured in TBU units) of the used burlulipase remain in the tablets and often even less than 60%. The extent of the loss of activity also depends on the processing conditions. At the same time burlulipase seems to be sensitive to some of the excipients used in the tablets. The loss of activity is so great that the use of such tablets as medicinal products is generally not possible. This also applies because the difference in the activity between two different batches can be very great.

Surprisingly, however, it could be shown that is is possible to produce solid pharmaceutical compositions containing burlulipase in the coating. In the production of the pharmaceutical composition according to the invention, surprisingly the burlulipase is not, or is only inactivated to a small extent. The activity is also reproducible in different batches. Moreover, the pharmaceutical composition according to the invention is surprisingly also stable when stored at room temperature. In the production of the compositions according to the invention the loss of burlulipase activity is so small that a practical use of the pharmaceutical composition according to the invention becomes possible.

The pharmaceutical composition according to the invention can be easily produced and allows storage at room temperature over a usual period for a medicinal product. Even when stored for several years, the activity of the lipases contained therein only decrease to a pharmaceutically acceptable extent. Artificial stabilisation of the lipase can also be dispensed with. The use of crystalline lipase becomes unnecessary. Cross-linking of the lipase is also not required. The pharmaceutical composition according to the invention is therefore preferably characterised in that the lipase contained therein is not chemically modified. Preferably it is also characterised in that the lipase contained therein is not present in crystalline form, i.e. it is amorphous. Particularly preferably the lipase is not chemically modified and is present in amorphous form. The embodiments in this paragraph apply in particular to burlulipase.

As pancreatin, in this invention any solid pharmaceutical preparations of pancreatin can be used. Particularly preferably the pharmaceutical composition according to the invention comprises a core that contains pancreatin onto which the coating is applied. This allows the use of the previously known medicinal products and their precursors as a core for producing the pharmaceutical composition according to the invention. In all forms of the embodiment, The proportion of lipase and in particular burlulipase can be adjusted by the layer thickness of the coating, and various quantity ratios between pancreatin and lipase, in particular burlulipase, can be set.

The pharmaceutical composition according to the invention can be free of gastric juice-resistant coatings. This is preferred. Gastric acid-resistant coatings are practically indispensable for conventional medicinal products containing pancreatin, as the lipase contained in pancreatin is broken down by the gastric juice. Gastric-juice resistant coatings are formulated so that they dissolve at the pH value usually prevalent in the duodenum and release the pancreatin. Paradoxically, the effect of pancreatin for the treatment of digestive disorders is therefore based on that fact that at least part of digestion functions smoothly, namely the pH regulation in the stomach and above all in the duodenum. However, this is not or not always the case in many patients. The duodenum often has too low a pH value, which means that the enzymes of the pancreatin are not released or not released completely or in time. Even the administration of pH regulating medications cannot always prevent this with adequate reliability. However, their administration is often associated with considerable side effects. The pharmaceutical composition according to the invention preferably contains at least one lipase which is stable on contact with the gastric acid. A gastric juice-resistant coating is therefore not necessary. The release of the lipase of the coating, and of the lipases, proteases and amylases of the pancreatin therefore ensures that breakdown already starts in the stomach, which promotes adequate digestion. The inactivation in the stomach of the lipases of the pancreatin can easily be compensated for by a larger quantity of the at least one lipase in the coating. The pharmaceutical composition according to the invention therefore guarantees an adequate supply of digestive enzymes also in patients with acid regulation disorders in the stomach and/or duodenum. Temporary overacidification of the stomach or strongly acidic or alkaline food also have no negative impact on the effect. The pharmaceutical composition according to the invention without a gastric juice-resistant coating can therefore resolve the drawbacks of pancreatin in connection with the acid content of the stomach and duodenum. As the applied bacterial lipase is acid-stable, it ensures sufficient lipolytic activity and makes the additional administration of medications for acid regulation unnecessary or reduces the need to administer them. Surprisingly, in the administration of the pharmaceutical composition according to the invention the additional administration of medications to inhibit gastric acid secretion can generally be dispensed with. In this way the sometimes severe side effects otherwise associated with these medicinal products can be prevented. In addition, the digestive enzymes can start working already in the stomach and thereby improve digestion.

The pharmaceutical composition according to the invention can, however, also have a gastric juice-resistant coating. This gastric juice-resistant coating can at the same time contain the at least one lipase. However, the at least one lipase can also be contained in a further coating or in both coatings. Forms of embodiment of the pharmaceutical composition according to the invention provided with a gastric juice-resistant coating exhibit a high degree of lipase activity and are stable when stored under the usual conditions.

In a further form of embodiment of the pharmaceutical composition according to the invention, a pancreatin-containing core is surrounded by at least one gastric juice-resistant coating, on which in turn a further coating is applied that contains the at least one lipase. This prevents any impairment of the activity of the lipase in the coating through incompatibilities with the ingredients of the core. Although in such a form of embodiment it can happen that in patients with acid regulation disorders in the stomach and/or duodenum the lipase of the pancreatin is not released, this can be easily compensated for by a sufficient dose of the lipase in the coating. Even in the event of a gastric acid secretion disorder this form of embodiment develops sufficient lipolytic activity. It brings about improved digestion, as the lipase of the coating is released already in the stomach. Preferably the lipase in the coating is a lipase, the lipolytic activity of which is stable against the simulated effect of gastric acid. Particularly preferably this is burlulipase.

As pancreatin-containing cores, in particular medicinal forms as already present in commercially available products can be used, for example the capsule filling material in the medicinal product “Cotazym®” by the company Cheplapharm Arzneimittel GmbH in Mesekenhagen, Germany as an example of pancreatin pellets without excipients provided with a gastric juice-resistant coating. Also suitable for example is the capsule filling material in the medicinal product “Kreon®” by the company Mylan Healthcare GmbH in Hannover, Germany as an example of pellets containing pancreatin and excipients provided with a gastric juice-resistant coating. Also suitable is the capsule filling material in the medicinal product “Pankreatan®” by the company Nordmark Arzneimittel GmbH & Co. KG in Uetersen, Germany, as an example of micro-tablets of pancreatin with excipients provided with a gastric juice-resistant coating. Another example of suitable pancreatin-containing cores is the medicinal product “Helopan®” by the company Nordmark Arzneimittel GmbH & Co. KG in Uetersen, Germany as an example of tablets with a gastric juice-resistant coating.

Examples of suitable pancreatin-containing cores without a gastric juice-resistant coating are the medicinal products “Eurobiol® 12500, granules” by the company Laboratoires Mayoly Spindler in Chatou, France, which are loose micro-tablets of pancreatin with excipients to be administered with a dosing spoon which are provided with a—however not gastric juice-resistant—film coating and “Viokace®” by the company Allergan USA, Inc. in Irvine (Calif.), USA, as an example of non-coated tablets.

Embodiments of the pharmaceutical composition according to the invention provided with a gastric juice-resistant coating exhibit a high degree of lipase activity and are stable when stored under the usual conditions. They bring about improved digestion as the lipase of the coating is released already in the stomach and, mixed with food, they can be administered to small children, infants and older people, as the partial destruction of the gastric juice-resistant coating has no influence on the activity of the lipase present in the coating.

It is advantageous if the pharmaceutical composition according to the invention therefore comprises a core made of pancreatin. The absence of excipients in the core increases the specific activity of the formulation and allows the administration of the same activity through formulations of smaller size. Also reduced through is this the impairment of the activity of the at least one lipase in the coating and in particular the burlulipase through incompatibilities with ingredients of the core. Such cores are described in EP 2 295 039 A1 for example. The core can take on various forms. It can, for example, be selected from the group consisting of tablets, micro-tablets, fast disintegrating micro-tablets, granulates, pellets and powders. Preferably a fast disintegrating (or dissolving) micro-tablet (FDMT)) is used as the core. Such formulations allow the rapid and complete release of the enzymes. This can take place already in the stomach or even in the mouth. Through dissolving the coated fast disintegrating micro-tablets, they can also be used to produce dissolutions, emulsions or suspensions which can be administered in liquid form, for example via feeding tubes. All forms of embodiment of the preparation according to the invention can of course be administered in this way. Liquid formulations of burlulipase are thermally unstable and can only be marketed in the form of cooled solutions with all the problems which an uninterrupted cool chain entails. The pharmaceutical composition according to the invention can be successfully used here in order to circumvent the storage of liquid formulations. Packaging is also simpler than with liquids.

Another preferred embodiment of the pharmaceutical composition according to the invention is characterised in that it contains a pancreatin with a reduced content of lipase and viruses. Porcine pancreas contains various viruses. The number of active viruses can be reduced through the action of heat and by other methods. In connection with such virus reduction or inactivation, a portion of the lipases of the pancreatin is also generally inactivated. Such pancreatin can ideally be used in the pharmaceutical composition according to the invention without running the risk that the lipolytic activity could be inadequate. The quantity of lipase in the coating is used in such formulations to replace the inactivated part of the pancreatin's lipases. At the same time a largely virus-free pharmaceutical composition is obtained.

Preferred is a pharmaceutical composition according to the invention which is characterised in that in its coating it exhibits a lipolytic activity in the range from 10,000 to 500,000 TBU/g. The enzymatic activity of the at least one lipase, preferably a bacterial lipase, particularly preferably burlulipase, in the coating of the pharmaceutical composition according to the invention as described herein is, in the case of multiparticulate formulations such as pellets or mini/micro-tablets, preferably 10,000 to 500,000 TBU/ and particularly preferably 50,000 to 400,000 TBU/g. Very particularly preferably in the case of mini/micro-tablets the range is 100,000 to 200,000 TBU/g, in the case of slightly larger pellets (in the range from 1.4 to 2.4 mm) in the range from 200,000 to 300,000 TBU/g, and in the case of slightly smaller pellets (in the range from 1.0 to 1.6 mm) in the range 225,000 to 375,000 TBU/g. The enzymatic activity of the at least one lipase, preferably a bacterial lipase, particularly preferably burlulipase, in the coating of the pharmaceutical composition as described herein is, in the case of monolithic formulations such as tablets preferably from 10,000 to 200,000 TBU/g, particularly preferably from 20,000 to 150,000 TBU/g and very particularly preferably from 50,000 to 100,000 TBU/g.

The core of the pharmaceutical composition according to the invention contains pancreatin, which in turn contains the three enzyme fractions of the lipases, amylases and proteases. With regard to this it is also preferred that both the respective content of the enzymatic activity (lipolytic activity, amylolytic activity, proteolytic activity) in the core as well as the purely numerical ratio of these enzymatic activities in the core in relation to each other—like for the pancreas enzymes usually in relation to units according to the monograph “Pancreas powder” of the European Pharmacopoeia (Ph. Eur.) , i.e. to Ph. Eur. units—lie in the respective ranges of the pancreatin-containing products available on the market. Examples of such products, whose pancreatin-containing formulations can by their nature be used as pancreatin-containing cores in the pharmaceutical composition according to the invention have already been cited above. Commercially available Pankreatan® 10,000 exhibits a lipolytic activity of 10,000 Ph. Eur. units, an amylolytic activity of 7,500 Ph. Eur. units and a proteolytic activity of 450 Ph. Eur. units. The corresponding contents in relation to capsule filling material—in this example micro-filmtablets—are in the range from approx. 52,000-62,500 Ph. Eur. units/g for the lipolytic activity, 39,000-47,000 Ph Eur. units/g for the amylolytic activity and 2,300-2,800 Ph. Eur. units/g for the proteolytic total activity.

The pharmaceutical composition according to the invention can, apart from the pancreatin and the at least one lipase in the coating or a plurality of lipases in the coating, also contain excipients. Excipients in the context of this application are all auxiliary substances that are usually used in pharmaceutical compositions. Active substances, in particular enzymes, are not excipients in accordance with this application. Suitable excipients are listed in the “Handbook of Pharmaceutical Excipients” of the “American Pharmaceutical Association”, for example.

The pharmaceutical composition according to the invention described herein can, in addition to the enzymes contained in the pancreatin and the further lipases contained in the coating, also contain further enzymes, in particular, further digestive enzymes. Enzymes that could be considered as digestive enzymes are in particular those selected from the group consisting of proteases and amylases. A pharmaceutical composition according to the invention which contains both protease and amylase also forms part of the present invention.

The pancreatin used in the pharmaceutical composition according to the invention can be any pancreatin in solid form, also in the form as described for example in the Ph. Eur. Pancreatin can be commerially acquired. A method of carefully obtaining the pancreatin is described in DE 32 48 588 A. A pancreatin produced in this way is preferred. A method of producing pancreatin micro-pellets is described in EP 0 583 726 A2. EP 0 436 110 A1 describes a method of producing spherical particles that contain pancreatin. In EP 2 295 039 B1 pancreatin pellets and micro-pellets are described which exclusively contain pancreatin. The pancreatin products described in these documents can be used in the pharmaceutical composition according to the invention. The core used in this invention can also be any known solid formulation of pancreatin.

The coating can exclusively consist of the at least one lipase. It can also exclusively consist of burlulipase. However, it is preferable if the coating comprises pharmacologically compatible excipients. Suitable excipients are listed in the “Handbook of Pharmaceutical Excipients” of the “American Pharmaceutical Association”, for instance. The coating can contain one or more excipients selected from the group consisting of binding agents, softeners, release agents, fillers, carrier materials, humectants, disintegrating agents and colouring agents. This list is not exhaustive and many other excipients known to a person skilled in the art can be used. Suitable pharmacologically compatible binding agents that can be present in the coating are, for example compounds selected from the group containing hydroxypropyl methylcellulose, polyethylene glycols, polyoxyethylene, polyoxyethylene polyoxypropyleine copolymers and mixtures thereof. This list is not exhaustive and and other binding agents known to a person skilled in the art can be used. Suitable colouring agents are for example food colourants, in particular food colourants which are described in the German drug colourant regulations. If a gastric acid-resistant coat is to be used, materials and methods known to a person skilled in the art can be used, whereby this applies in particular to the aforementioned materials and methods. Such materials and methods are also described in EP 2 295 039 B1 in paragraphs [0028] to [0033].

### Medicinal Product

Another aspect of the invention is a medicinal product comprising a pharmaceutical composition according to the invention. Preferably, this is a medicinal product for the prevention and/or treatment of digestive disorders, in particular exocrine pancreatic insufficiency and particularly preferably a medicinal product for the prevention and/or treatment of exocrine pancreatic insufficiency in patients with cystic fibrosis, in infants and children as well as elderly and bed-ridden patients.

### Method of Producing the Pharmaceutical Composition

A further aspect of the present invention is a method of producing the pharmaceutical composition according to the invention comprising the steps:
Provision of pancreatin,
Provision of a coating composition for the coating containing at least one lipase,
Coating of the pancreatin with the coating composition for the coating.

Preferably the at least one lipase in the coating composition is a microbial lipase, particularly preferably a bacterial lipase and most preferably at least one lipase in the coating composition is burlulipase. Suitable forms of embodiment of the pancreatin and the ingredients of the coating and thus the coating composition are described above. Preferred as the coating composition is a solution of the components needed for the coating in water. Preferred is a method according to the invention in which the pancreatin is provided in the form of a core from a fast disintegrating micro-tablet containing pancreatin and the coating composition is a solution of burlulipase, hydroxypropyl methylcellulose and possibly other excipients in water.

The coating can be produced by means of known techniques, for example in a fluidised bed processor with a Wurster column or a ball coater. For a coating the pancreatin is introduced into the apparatus and a previously produced mixture of the at least one lipase for the coating and the excipients and possibly a solvent is sprayed in.

As burlulipase and other lipases can be inactivated through the effect of pressure, higher pressures should be avoided as far as possible. Preferred therefore is a production method according to the invention for the pharmaceutical composition according to the invention in which pressing procedures after adding the at least one lipase for the coating and in particular the burlulipase are dispensed with. Particularly preferred are production methods according to the invention for the pharmaceutical compositions according to the invention in which all mechanical pressures effects for compressing or solidifying the pharmaceutical composition according to the invention after adding the at least one lipase for the coating and in particular the burlulipase are dispensed with.

### EXAMPLES

### Analysis Methods:

The analytical determination of the lipolytic activity takes place by way of the so-called tributyrine assay according to Erlanson, Ch. & Borgstrom, B.: “Tributyrine as a Substrate for Determination of Lipase Activity of Pancreatic Juice and Small Intestinal Content”; Scand. J. Gastroent. 5, 293-295 (1970). The lipolytic activity determined by the so-called tributyrine assay is synonymously indicated in TBU units (TBU u., sometimes abbreviated to TBU) wherein the spellings (with/without abbreviation full stop, with/without hyphen as well as with/without a space) sometimes vary greatly in the scientific literature. An enzymatic activity of 1 unit (1 enzyme unit) corresponds to a material conversion of 1 μmol substrate per minute.

### Example

### Production of a Pharmaceutical Composition According to the Invention

Into 24.8 parts by weight of a 10.5% solution of hydroxypropyl methylcellulose (type C606 from the company “HARKE Germany Services GmbH & Co. KG”, in Mülheim an der Ruhr,Germany) in water, are stirred 75.2 parts by weight of a solution of burlulipase in water with a TBU activity of 64650 TBU/ml. The pH value is adjusted to 7.5 with 3% sodium hydroxide solution.

450 g of spherical pancreatin micro-pellets consisting of pancreatin produced by the method according to claim 1 of EP 2 295 039 B1 with a mean particle diameter of around 0.6 mm are coated in a Wurster spray coating system of type Glatt-GPCG-5 with a Wurster column with 4,500 g of the solution of burlulipase. The following parameters are used: atomiser air pressure 1.5 bars, air quantity 50-55 m3/h, inlet air temperature 47.3-49.7° C., product temperature 30.6-31.8° C., spray rate 4.9-6.0 g/min, spray duration 160 mins. The yield is 96.7%.

The loss of activity (in TBU) of burlulipase in the coated micro-pellets through the spraying process is 22.2%. This includes the loss through the enzyme adhering to the apparatus. The corrected loss of activity through inactivation of the burlulipase is 20.8%. After storage at 25° C. over 8 months in polyethylene bags no further changes in the activity can be detected. A repeat of the experiment with pancreatin micro-pellets produced in accordance with example 2 of EP 0 436 110 A1 produces very similar results. If pure solutions of burlulipase in water (76,000 TBU/ml) are used for coating, the losses of activity are 17.4% through the spraying process and 13.2% corrected loss through inactivation of the burlulipase. These results are reproducible and the products are suitable for use as medicinal products.

### Comparative Example

5,000 g magnesium stearate are added via a 0.25 mm sieve to 495.0 g of a microcrystalline cellulose (proprietary name: “Avicel PH101” available from “FMC Corporation”, Philadelphia, USA) and mixed in a Zoller gravity mixer for 10 minutes at 25 rpm. 300 mg of the thus produced carrier mixture are mixed with 150 ul of a solution of burlulipase in water with an activity of 50,050 TBU/ml and worked in with a spatula until the solution is fully incorporated. The resulting mixture is dried in a vacuum drying oven at room temperature (<25° C.) and 50 mbar for 1.5 hours. The thus obtained mixture is pressed into tablets by means of a Korsch EKO eccentric press and stamping tool with a diameter of 10 mm, dragée-convex with a pressing force of 21.0 kN. The finished tablets are placed in water and the TBU activity is determined. The burlulipase activity loss (TBU) is 41.5%. The present pharmaceutical composition is thus superior to normal tabletting. If the burlulipase is added to the carrier mixture in the form of a solid lyophilisate, similar losses of activity are obtained.

1. Pharmaceutical composition comprising pancreatin, having a coating that contains at least one lipase, wherein the pharmaceutical composition comprises a core which contains pancreatin onto which the coating is applied, characterised in that the at least one lipase used in the coating is a lipase different from the lipases present in the pancreatin.

2. Pharmaceutical composition according to claim 1, characterised in that the activity of the at least one lipase is stable against the in vitro simulated effect of gastric acid.

3. Pharmaceutical composition according to any one of claim 1 or 2, characterised in that the at least one lipase is a bacterial lipase.

4. Pharmaceutical composition according to any one of claim 1, 2 or 3, characterised in that the at least one lipase is burlulipase.

5. Pharmaceutical composition according to any one of claims 1 to 4, more particularly according to claim 4, characterised in that it does not have a gastric juice-resistant coating.

6. Pharmaceutical composition according to any one of claims 1 to 5, characterised in that it comprises a core that is selected from the group consisting of tablets, micro-tablets, fast disintegrating micro-tablets, granulates, pellets and powders.

7. Pharmaceutical composition according to any one of claims 1 to 6, characterised in that it comprises a core that is a fast disintegrating micro-tablet.

8. Pharmaceutical composition according to any one of claims 1 to 7, characterised in that the coating comprises burlulipase and a binding agent.

9. Pharmaceutical composition according to any one of claims 1 to 8, characterised in that the coating comprises burlulipase and hydroxypropyl methylcellulose

10. Pharmaceutical composition according to any one of claims 1 to 9, characterised in that in its coating it exhibits a lipolytic activity in the range from 10,000 to 500,000 TBU/g.

11. Medicinal product comprising a composition according to any one of claims 1 to 10.

12. Medicinal product according to claim 11 for the prevention and/or treatment of digestive disorders.

13. Medicinal product according to any one of claim 11 or 12 for the prevention and/or treatment of exocrine pancreatic insufficiency in patients with cystic fibrosis.

14. Method of manufacturing a pharmaceutical composition according to any one of claims 1 to 10, comprising the steps:
Provision of pancreatin,
Provision of a coating composition for the coating containing at least one lipase,
Coating of the pancreatin with the coating composition for the coating.

15. Method according to claim 14, characterised in that the pancreatin is provided in the form of a pancreatin-containing fast disintegrating micro-tablet and the coating composition for the coating is a solution of burlulipase, hydroxypropyl methylcellulose and possibly other auxiliary substances in water.
